(19) United States
(12) Patent Application Publication
(10) Pub. No.: US 2024/0127936 A1
(43) Pub. Date: Apr. 18, 2024

(54) APPARATUS, METHODS, AND SYSTEMS FOR GENERATING PERSONALIZED RESPONSIVE ASSISTANCE FOR PERSONS NEEDING ASSISTANCE BY A SET OF PERSONS PROVIDING ASSISTANCE WHO ARE PERSONALLY KNOWN BY THE PERSON NEEDING ASSISTANCE

(71) Applicant: My Friendly Eyes Inc., Pinellas Park, FL (US)

(72) Inventors: Douglas George Towne, Pinellas Park, FL (US); John Medcalf, Valley Center, CA (US); Michael Wilhelm Hartman, Cibolo, TX (US); Corey Saur, Pinellas Park, FL (US)

(21) Appl. No.: 18/227,764

(22) Filed: Jul. 28, 2023

Related U.S. Application Data
(60) Provisional application No. 63/415,548, filed on Oct. 12, 2022.

(51) Int. Cl. G16H 40/20

(52) U.S. Cl. CPC G16H 40/20

(57) ABSTRACT

A computer implemented method for generating personalized responsive assistance for a person needing assistance (PNA) is disclosed. The method begins with the system receiving a PNA's contact data and a trust group manager's (TGM) contact data. The system then sends a request to the TGM for association of TGM's device with PNA's device. The system then receives approval and proceeds with storing an indicator of the association of the devices. The TGM then sends the system a person providing assistance's (PPA) data. The system will then send an invite to the PPA, to join a trust group associated with the PNA. After the PPA accepts the invite to join the PPA group, the system will receive a request for an audio/visual connection between the PNA and the PPA. As the PNA is in need of assistance, an audio/visual connection will be permitted allowing the PPA to help the PNA.

### REFERENCE TO RELATED APPLICATIONS

This application is a U.S. Non-Provisional application which claims the benefit of the filing date of U.S. Provisional Application Ser. No. 63/415,548 titled “APPARATUS, METHODS, AND SYSTEMS FOR GENERATING PERSONALIZED RESPONSIVE ASSISTANCE FOR PERSONS NEEDING ASSISTANCE BY A SET OF PERSONS PROVIDING ASSISTANCE WHO ARE PERSONALLY KNOWN BY ME PERSON NEEDING ASSISTANCE” and filed Oct. 12, 2022 and the subject matter of which is incorporated herein by reference.

### CROSS-REFERENCES

Not applicable.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISK

Not applicable.

### TECHNICAL FIELD

The present disclosure relates to the field of software applications for assisting people in need of assistance.

### BACKGROUND OF THE INVENTION

Users with needs resulting from intellectual/cognitive, physical and sensory disabilities such as blindness, autism and elders often require assistance with everyday tasks such as navigation or assistance with written documents. In the past, software applications such have provided services for individuals with visual impairments by utilizing agents that can see through the back/rear facing camera of the user's smart phone. Additionally, in the past, services have provided assistance with documents such as billing statements or assisting in making online purchases. However, such services can be costly and may be well beyond the financial abilities of many across the disability and elder community.

Additionally, services used for helping people in the disability and elder community require over five million volunteers to support nearly 400,000 people who are blind and low vision across more than 150 countries. However, such volunteers, while in many cases helpful, are not bonded or trained to a level of support to provide adequate care and are clearly not appropriate for assistance with documents such as financial statements or assisting in making online purchases.

The current state of the art is that many services need to provide assistance to people with cognitive disabilities, or elders, which takes a deeper level of training, understanding and engagement. Finding and maintaining trained supporters is a challenging effort that often leads to situations in which their users are stuck without support for periods of time. The length of time the service is unavailable is detrimental if you are the person who needs assistance immediately.

In addition, many of the existing prior art services require massive infrastructure to maintain support by connecting users to each other. There are few existing software applications that provide support for users with intellectual/cognitive disabilities. Some existing software applications exist to allow caregivers to monitor the location of elders or users with intellectual/cognitive disabilities. However, none provide the users the ability to find support quickly and easily when in need.

Software applications such as FaceTime® allow users to call someone and provide them access to their camera. However, FaceTime® is not available across platforms (e.g., to allow iOS® users to call contacts that use Android® phones) and the process of determining which contacts to use can be a complicated task, particularly for individuals with intellectual, developmental, and/or sensory disabilities. Additionally, operating FaceTime® might pose significant challenges for many people as well.

As a result, there exists a need for improvements over the prior art and more particularly for a more reliable and efficient way of providing a solution and services to people of the disability and elder community with a means for providing assistance with everyday tasks.

### BRIEF SUMMARY OF THE INVENTION

A computer implemented method, executable on at least one processor of a first computing device, for generating personalized responsive assistance for persons needing assistance (“PNA”) by a set of persons providing assistance (“PPA”) who are personally known by the PNA is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a computer implemented method, executable on at least one processor of a first computing device, for generating personalized responsive assistance for persons needing assistance by a set of PPAs who are personally known by the PNA is disclosed. The method begins with a first computing device, being the system, receiving a first message including a PNA contact data and a trust group manager's (“TGM”) contact data. The system will then generate a PNA user record and store said record in a connected database. The system then proceeds to send a message to a third computing device, associated with a TGM, a request for approval to associate said device with the second computing device of the PNA user. Next, the system will receive approval from the third computing device to associate said device with the second computing device of the PNA. Once the system associates the third computing device with the PNA's device, the system will store a unique identifier indicating the association between the second computing device of the PNA and the third computing device of the TGM. Now the TGM will send the system a message having at least one person providing assistance's (“PPA”) data. The system will then send an invite to the fourth computing device, associated with at least one PPA, to join a trust group associated with the PNA user record. After the fourth computing device accepts the invite to join the PPA group, the system will receive from the second computing device associated with the PNA, a request for an audio-visual connection between the PNA's second computing device and the PPA's fourth computing device. As the PNA is in need of assistance, an audio-visual connection will be caused allowing the PPA to help the PNA.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the disclosure and together with the description, explain the principles of the disclosed embodiments. The embodiments illustrated herein are presently preferred, it being understood, however, that the disclosure is not limited to the precise arrangements and instrumentalities shown, wherein:

FIG. 1 is a diagram of an operating environment that supports a computer implemented method, executable on one or more processors, for generating personalized responsive assistance for persons needing assistance by a set of persons providing assistance who are personally known by the person needing assistance, according to an example embodiment;

FIG. 2A is a flow chart showing the steps taken in a computer implemented method, executable on one or more processors, for generating personalized responsive assistance for persons needing assistance by a set of persons providing assistance who are personally known by the person needing assistance, according to an example embodiment;

FIG. 2B is a continuation of the flow chart showing the steps taken in a computer implemented method, executable on one or more processors, for generating personalized responsive assistance for persons needing assistance by a set of persons providing assistance who are personally known by the person needing assistance, according to an example embodiment;

FIG. 3 is a diagram showing the data flow as the system receives a first message, containing a person needing assistance's data and a trust group managers data, and sends a second message containing a request for approval to associate the trust group manager with the person needing assistance, according to an example embodiment;

FIG. 4 is a diagram showing the data flow as the system receives a third message containing an approval for association and a fourth message containing a person providing assistance's data, sends a fifth message containing an invite to join a trust group, and receives a sixth message containing a person providing assistance's availability profile and acceptance, according to an example embodiment;

FIG. 5 is a diagram showing the data flow as the system receives a seventh message containing a request for audio-visual connection between the person needing assistance's device and the person providing assistance's device, sends, to the person providing assistance, an eighth message containing the request, and receives a response causing a connection, according to an example embodiment;

FIG. 6 is a diagram showing a first subset group of persons providing assistance and a second subset group of persons providing assistance, according to an example embodiment;

FIG. 7 is a flow chart showing the steps taken when attempting to establish an audio-visual connection between the person needing assistance and a person providing assistance, according to an example embodiment;

FIG. 8 is a diagram showing the data flow when attempting to establish an audio-visual connection between the person needing assistance and a person providing assistance, according to an example embodiment;

FIG. 9A is a diagram showing the data flow as the system receives a request to connect the trust group manager's device, the person needing assistance's device, and the person providing assistance's device to each other, according to an example embodiment;

FIG. 9B is a diagram showing the data flow as the system receives a request to connect the trust group manager's device and the person providing assistance's device to each other, according to an example embodiment;

FIG. 10A is an illustration of a user interface on a person needing assistance's device displaying a button to call for help, according to an example embodiment;

FIG. 10B is an illustration of a user interface on a person needing assistance's device displaying controls while utilizing the front facing camera, according to an example embodiment;

FIG. 10C is an illustration of a user interface on a person needing assistance's device displaying controls while utilizing the rear facing camera, according to an example embodiment; and

FIG. 11 is a block diagram of a system including an example computing device and other computing devices, according to an example embodiment.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions, or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims. The disclosed embodiments improve upon the problems with the prior art by providing apparatus, methods and systems that guarantee the PPAs have been vetted by the PNA or the PNA's guardian or TGM. The system ensures that the PPAs have been vetted by providing methods for the TGM computing device to authenticate the PPA and PPA computing devices so that each of the PPAs assisting the PNA is personally known by the PNA. The system also improves over the prior art by establishing geographic and time and date constraints (or a profile for the PNA) for the PNA and PNA computing device such that if those constraints are not satisfied, then the system and methods will send a message to PPA computing devices notifying the PPA computing devices that the PPA is not within a predetermined geographic area.

Additionally, the system allows for calendar type constraints which will make it easy for the notifications to be paused easily. Additionally, methods may be included that allow notifications to be paused within the PNA computing device is within close proximity to a PPA computing device. The invention also improves over the prior art by providing a simple and easy to use user interface on the PNA computing device so that the PNA can easily make a call to one of the PPA computing devices. The invention also improves over the prior art by, after receiving from the PNA computing device a call for assistance, the invention will call the PPA computing devices in according to set a predetermined parameters until a PPA answers the phone. The system is also an improvement over the prior art because it includes methods for which the TGM computing device or another computing device associated with the system, arranges the PPA user records into a priority notification contact list. The priority notification contact list may be the order by which the invention attempts to call or contact a PPA computing device when the PNA needs help. It is understood that as a last resort, one of the PPA and TGM computing devices may be a professional organization such as a first responder, police, etc.

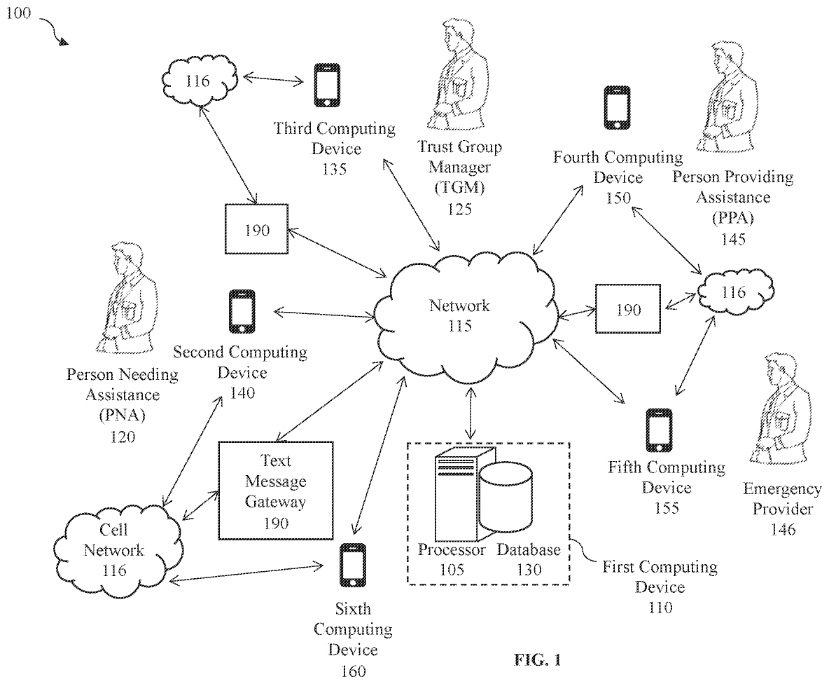

Referring now to the Figures, FIG. 1 is a diagram of an operating environment 100 that supports a computer implemented method, executable on one or more processors 105, for generating personalized responsive assistance for persons needing assistance by a set of persons providing assistance who are personally known by the person needing assistance. The operating environment 100 may comprise computing devices 110, 135, 140, 150, 155, and 160 all of which may communicate with processor 105 via a communications network 115.

The first computing device 110 may be defined as the system. The first computing device may include a processor 105 and a database 130. The second computing device 140 is referred to as the computing device that may be used in association to the PNA 120. The PNA computing device may be configured to provide a simple user interface (further explained below) so that the PNA can easily use the device if the person has significant physical or cognitive challenges. As mentioned above, a PNA 120 may be a person with physical or cognitive disabilities. The third computing device 135 is referred to as the computing device that may be used in association with the TGM 125. As mentioned above the TGM or trust group manager may be a person who is trusted by the PNA to manage or contact people able to help the PNA when in need. However, it is understood that in other embodiments, the system may not include a TGM. In some embodiments, the PNA or PPA may take on the role of a TGM. The TGM 125 may be a person that may act as a guardian for the PNA such as a parent, family member, teacher, guardian etc. It is understood that the TGM may be the TGM for one of more PPAs. The fourth computing device 150 is referred to as the computing device in association with the PPA or person providing assistance. The PPA 145 may be a person personally known to the PNA that has agreed to a provide assistance to the PNA. The fifth computing device 155 is referred to as the computing device in association with an emergency responder 146. All computing devices are defined in further detail below.

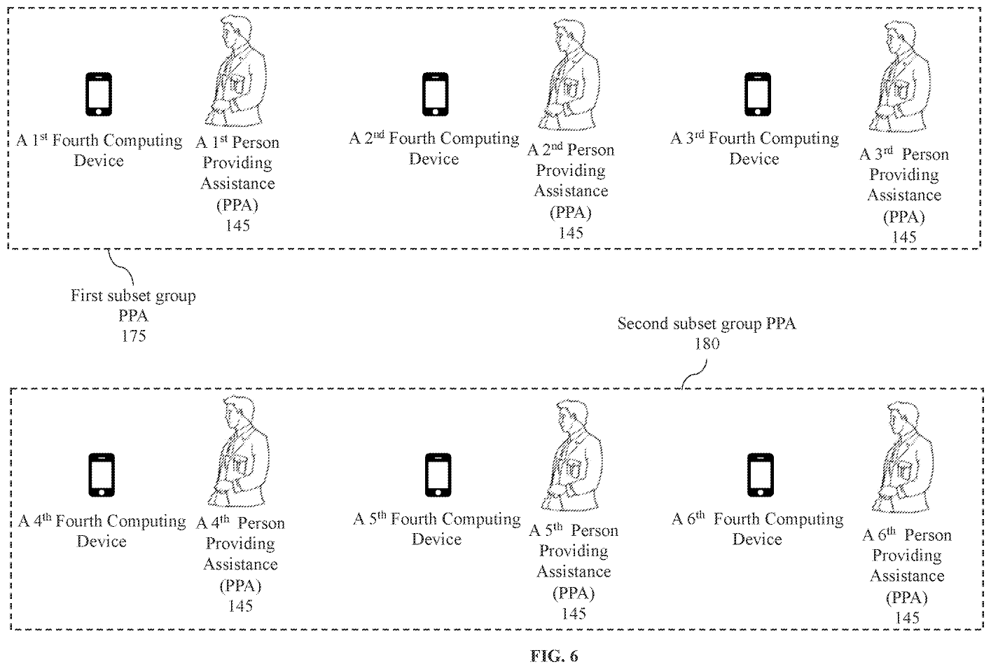

Although FIG. 1 displays one computing device for each user, it is understood that many users and computing devices may be involved in the system. For example, FIG. 6 illustrates six PPAs being used according to that example embodiment. Having multiple PPAs increase the probability of a PPA being available at the time of need of the PNA. In other embodiments, the system may include more than one TGM or TGM devices. In some embodiments, it is important that the PPA and PNA have access to the TGM in case of emergencies. Having a primary TGM along with one or two back up TGMs may ensure that at least one TGM is present and available at all times. The third computing device is also shown to be singular in FIG. 1, however in other embodiments, the TGM may register multiple devices with the system as well. For example, the TGM may want a large tablet, best used on group calls or video calls, to be registered while also having their mobile phone available to the system for times when the TGM does not have access to the large tablet.

Computing devices 110, 135, 140, 150, 155, and 160 may comprise any computing devices, such as integrated circuits, printed circuit boards, processors, ASICs, PCBs, cellular telephones, smart phones, tablet computers, laptops, and game consoles, for example. Computing devices 110, 135, 140, 150, 155, and 160 may be connected wirelessly to the communications network 115. Communications network 115 may include one or more packet switched networks, such as the Internet, or any local area networks, wide area networks, enterprise private networks, cellular networks, phone networks, mobile communications networks, or any combination of the above. In one embodiment, computing devices 110, 135, 140, 150, 155, and 160 are a programmable logic controller or PLC.

Processor 105 includes a software engine that delivers applications, data, program code and other information to networked computing devices 110, 135, 140, 150, 155, and 160. The software engine of processor 105 may perform other processes such as transferring multimedia data in a stream of packets that are interpreted and rendered by a software application as the packets arrive. FIG. 1 further shows that server/processor 105 includes a database 130 or repository, which may be a relational database comprising a Structured Query Language (SQL) database stored in a SQL server or a database that adheres to the NoSQL paradigm. Devices 110, 135, 140, 150, 155, and 160 may also each include databases.

The database 130 may serve sensor data, as well as related information, used by processor 105 and devices 110, 135, 140, 150, 155, and 160 during the course of operation of the invention. For example, such computing devices may include a sensor or position locating element for providing location data, which includes the geographic position of the mobile phone, for determining if the mobile device is within a certain predetermined geographic area or within the proximity of other mobile devices. Such sensor or position locating element may comprise technology such as GPS transceiver, GPS technology, a wireless communication element, such as WIFI, Bluetooth, NFC etc. Additionally, each of the computing devices may include a sensor or position locating element that provides accelerometer data, such as speed, velocity, acceleration, deceleration of the mobile phone, or include other technologies for determining if each mobile device is moving similar to how a motor vehicle is moving. In one embodiment, the data provided by the plurality of mobile devices is provided to processor 105 via TCP/IP and/or HTTP over communications network 115.

Devices 110, 135, 140, 150, 155, 160 and processor 105 may each include program logic comprising computer source code, scripting language code or interpreted language code that perform various functions of the present invention. In one embodiment, the aforementioned program logic may comprise program module 1107 in FIG. 11. As mentioned above, it should be noted that although FIG. 1 shows only one device 110, 135, 140, 150, 155, and 160 and one processor 105, the system of the present invention supports any number of computing devices, servers connected via communications network 115. Also note that although processor 105 is shown as a single and independent entity, in one embodiment, processor and its functionality can be realized in a centralized fashion in one computer system or in a distributed fashion wherein different elements are spread across several interconnected computer systems.

Various types of data may be stored in the database 130 of processor 105. For example, the database may be the configured to store user data of each user (PPA, PNA, TGM, Emergency Provider) in a user record. In one embodiment, the PNA user data may include PNA user identifying information or contact data such as a username, age, email address, disability information, medical information, family history/family information, emergency contact data, personal preferences, common places or areas of interest, and a PNA computing device identifier. PNA user data may also include GPS position data, emergency contact information, personalized messages to be sent to incoming callers, parameters for determining when to send notifications and to receive notifications, geographic constraints, calendar constraints, as well as many other parameters that are within the spirit and scope of the present invention. In one embodiment the geographic constraints will establish a geofence for the PNA computing device.

In one embodiment, PPA user data may include PPA user identifying information such as a username, age, email address and a PPA computing device identifier. PPA user identifying information may include a token or other data element identifying that the PPA computing device is an authenticated device and personally known to the PNA computing device and PNA user. PPA user data may also include a PPA availability profile for when the PPA is available for communicating with the PNA. For example, the availability profile may include days and times that the PPA is available to provide assistance to the PNA and able to receive calls from the PNA, geographic availability/limitations of when PPA is able to provide assistance to the PNA or receive calls from the PNA. Additionally, other types of data and other parameters that are within the spirit and scope of the present invention may be stored in the PPA user record. The information in the PPA user record may also be stored in the PNA user record.

A TGM user record may also be stored on database 130 on in a TGM user record or within the PNA user record. TGM user record may include TGM user identifying information or contact data such as a username, age, email address and a TGM computing device identifier. TGM user identifying information may include a token or other data element identifying that the TGM computing device is an authenticated device and personally known to the PNA computing device and PNA user. The TGM user record may also include other information that is within the spirit and scope of the present invention. The TGM user record may also include the PNA's trust group app settings, PPA onboarding, training, and interactions with assisting services (police, health, 911, etc.). Additionally, a TGM may be include data that makes the TGM a fiduciary for the payment of fees due the owner of the app and for extracting and presenting data captured by the app. The database 130 may include rules that defining the algorithms presented below. FIG. 1 further shows a text message gateway 190, which may be a third-party entity that provides text messaging services (including both SMS and MMS messages) to processor 105. The text message gateway 190 and devices 110, 140, 135, 150, 155, and 160 are coupled with cellular network 116 (in addition to network 115), which is a mobile phone network including a wireless communications network of transceivers. In other embodiments, the text message gateway and cellular network 116 may be included in network 115.

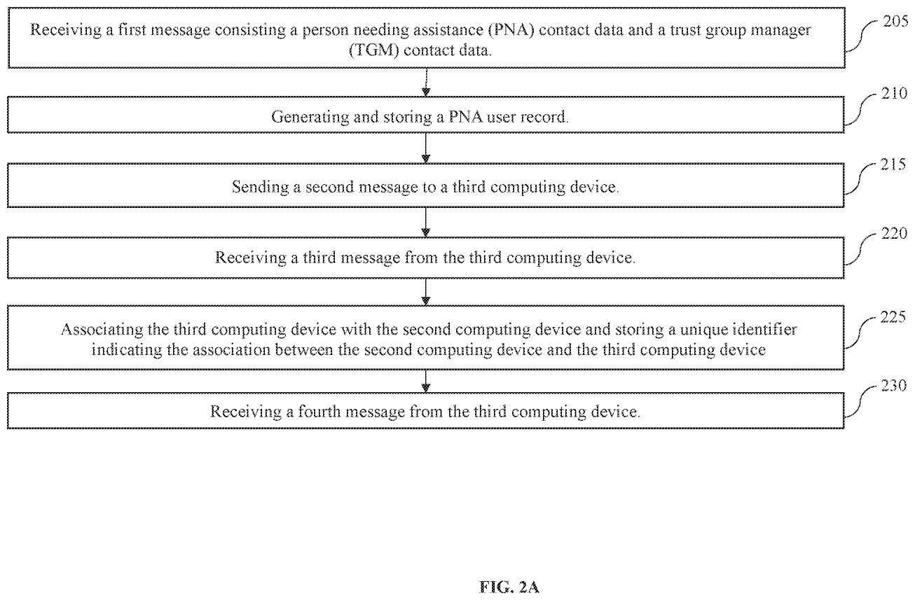

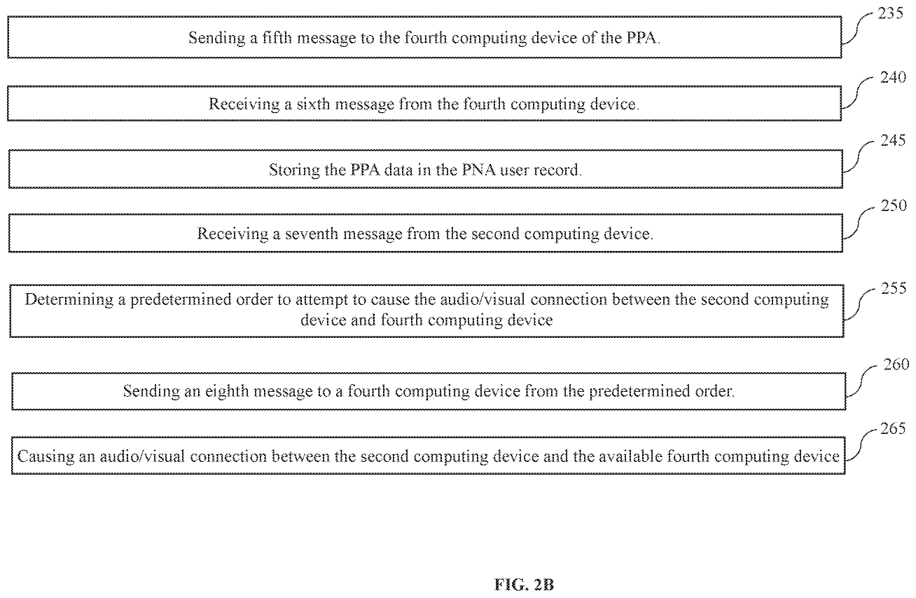

FIGS. 2A and 2B is a flow chart showing the steps taken in a computer implemented method, executable on one or more processors, for generating personalized responsive assistance for persons needing assistance by a set of persons providing assistance who are personally known by the person needing assistance. The method begins with step 205.

Step 205 includes receiving, with a transceiver of the first computing device 110, over a communications network 115, a first message 305 including a person needing assistance's (PNA) contact data 310 for a PNA 120 and trust group manager (TGM) contact data 315 of a TGM 125. A transceiver may generally be defined as a device or module that transmits and receives signals in a communication system. More specifically, the transceiver in the disclosed system sends and receives the plurality of messages and data being sent to and from the computing devices 110, 135, 140, 150, and 155. The transceiver may take various forms, for example in the context of wired connections, such as desktop computers, an ethernet transceiver may be used. However, the most likely type of transceiver used in the disclosed invention may be a wireless transceiver. Wireless transceivers are used in wireless communication systems such as Wi-Fi, Bluetooth, or cellular networks. Many of the computing devices may likely be cell phones, however, other computing devices may also be used and may require other forms of transceivers to communicate.

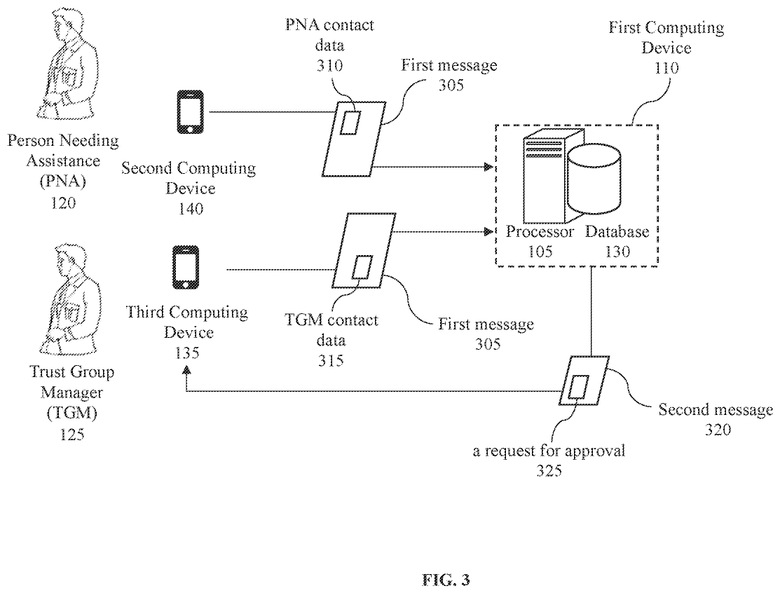

As mentioned above, the first computing device may be referred to as the system or system's processor. As shown in FIG. 3, the first message 305 is sent from the second computing device 140 of the PNA 120, and the third computing device 135 of the TGM 125, to the first computing device 110. In other embodiments, the first message may be sent from only one device containing both the PNA data and the TGM data. Furthermore, in other example embodiments, a third party such as a system manager may also start the process. For example, a PNA or TGM may decide to hire an experienced and trained person to manage the software disclosed. In such an embodiment, all parties of the system may provide said person with all the required or necessary data, and in return, the third party would input the data directly to the system or first computing device 110 prepping the system to function properly. In other example embodiments, there may be a plurality of computing devices sending the system first messages containing personal data to be stored within the connected database.

The first message 305 may include PNA contact data 310 and TGM contact data 315. PNA contact data may include the PNA's first name, last name, nickname, home address, phone number, disability data, usual daily routines, vehicle information, pet or service animal information, doctor contact or medical information, family and friends contact info, work schedule, etc. The TGM contact data 315 may include the TGM's first name, last name, nickname, home address, work schedule, phone number, availability profile, etc. Other information may also be provided and included in the PNA and TGM contact data. After the first message, containing the PNA and TGM contact data, is received by the first computing device 110, the method continues to step 210.

Step 210 includes generating a PNA user record for the PNA user and storing, in the connected database 130, the PNA user contact data 310. In addition to the PNA contact data, the PNA user record also includes at least one TGM-indicated advertising preference. The TGM-indicated advertising preference may generally be described as a system or method for enabling the TGM to customize and control the types of advertisements 400 they receive within the software application. This feature can benefit several parties involved within the system.

In one example embodiment, this preference may allow the TGM to decide which users the ads are displayed to such as PPAs rather than PNAs. For example, PNAs that are seeking assistance are often in a state of actively seeking guidance, advice, or solutions to their needs or problems. This vulnerable and receptive mindset may make the PNA more susceptible to consumer purchasing influence. Therefore, allowing the TGM to decide what type of ads or how many ads are displayed on the PNAs device, helps to protect the PNA from unnecessary influence. In other embodiments, the TGM-indicated advertising preference may benefit the advertisers. For example, by allowing a user to specify their preferences such as preferred product categories, content relevance, frequency, and presentation format, the system will likely enhance user engagement and optimize the overall advertising experience within the software application. In other embodiments, the PPA or PNA may also have advertising preferences stored within the user record. The system will use these preferences when displaying a plurality of advertisements based on the plurality of ad requests, on the fourth computing device of the PPA, or in other embodiments on any device within the system. In other embodiments, the system may take the PNA user preferences, in regard to the type of products or services of interest, and display said products or services to the TGM or PPA. Said example embodiment may benefit the users involved in such situations where the PNA is being celebrated and the PPA and TGM are shopping for gifts. In other embodiments, the PPA, PNA, and TGM may fill out a survey or questionnaire such that the system may find similar products or services involved with the interests recorded on the survey.

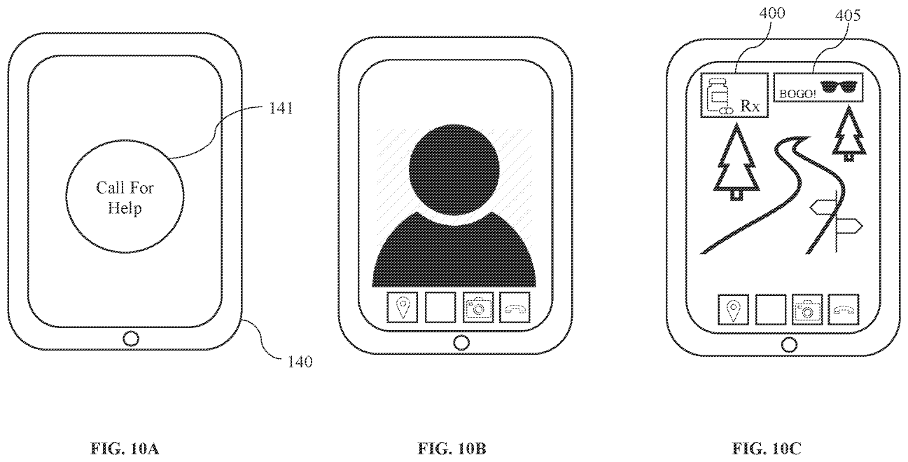

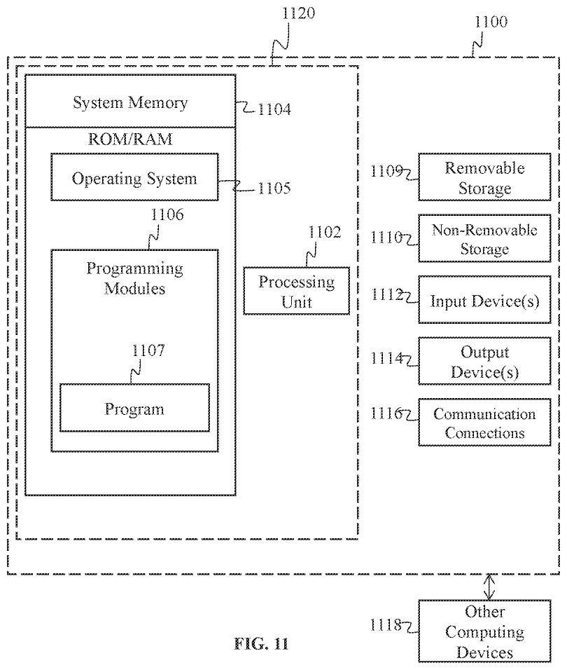

In some embodiments, the TGM or PPA may prefer to receive ads relating to PNA attributes such as medication or product preferences. For example, the users may all attend an occasion for the PNA such as a birthday party. By having access to the PNA's ad preferences 400, the TGM or PPA may now receive similar ads relate to the wants and needs of the PNA. In other embodiments, the TGM or PPA may prefer to receive both ads based upon the PPA or TGM attributes as well as the PNA attributes. FIG. 10C is an example of a user interface of a computing device displaying two ads at the top of the display while still allowing the user to use said app. FIG. 10 C includes an ad relating to or derived from the PNA's preferences 400 and an ad relating or derived from to the TGM or PPA's preferences 405. In some embodiments, the interface may label which ads are based on whose preferences.

The first computing device 110 may generate the user record by first creating a table defining a schema or structure for storing the record. Then computing device 110 may organize the data into the structured format/table and send the record to be stored in a specific place within the database to be easily accessed when needed. Other methods of generating and storing a user record within a database may also be used and are within the spirit and the scope of the present invention. After the PNA user record is generated and stored in the connected database, the method continues to step 215.

Step 215 involves the first computing device 110 sending, over the communications network 115, to a third computing device 135 associated with the TGM 125, a second message 320 comprising a request 325 for approval to associate the third computing device of the TGM user with the second computing device 140 of the PNA 120. The data flow during this step is illustrated in FIG. 3 for reference. The request may be displayed across the user interface in the form of a text message, push notification, email, survey, phone call, app-based request, etc. The association of the devices may generally be defined or described as the act, or process of, establishing a connection or relationship between two devices in order to facilitate communication, data exchange, or collaboration. The association of the third computing device with the second computing device is necessary for creating an audio-visual connection between the two. The TGM may approve of the request by pressing a button displayed by the user interface, saying “YES” to the phone, sending a text via short messaging services (“SMS”), etc. Other forms of requests and responses to the requests may also be used and are within the spirit and the scope of the present invention. After the first computing device 110 has sent the second message 320 containing the request for approval to associate with the third computing device 135, the method continues to step 220.

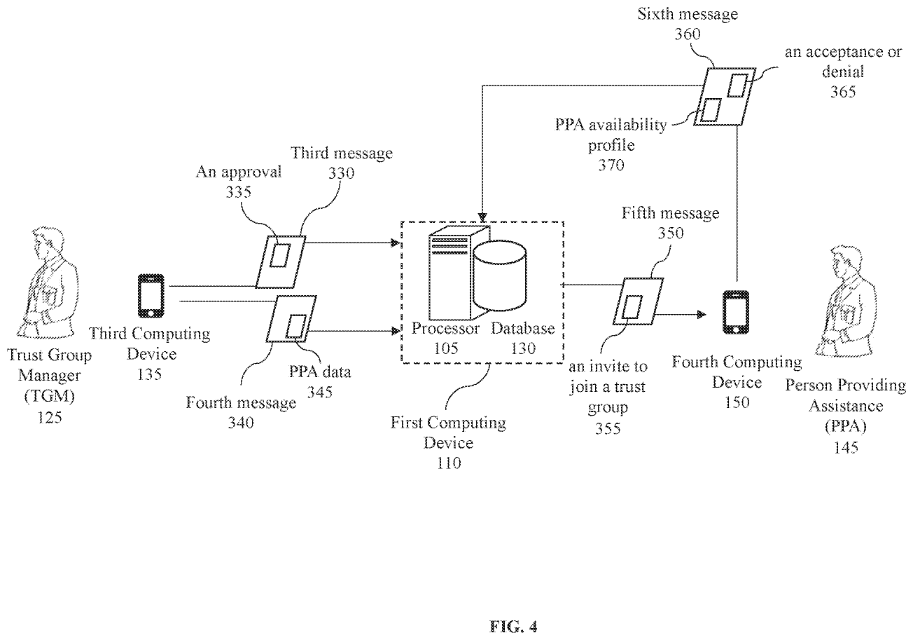

Step 220 includes receiving, over the communications network 115, from the third computing device 135, a third message 330 comprising an approval 335 of the request to associate the third computing device 135 of the TGM 125 with the second computing device 140 of the PNA 120. The data flow during this step is illustrated in FIG. 4 for reference. The messages used in the present system may also be referred to as data packets. These messages are discrete chunks of information that carry both the data being transmitted and the necessary control information for reliable delivery. As mentioned above, the approval may be in the form of a text message, voice memo, etc. After the first computing device, 110 receives approval 335, the method continues to step 225.

Step 225 occurs in response to the third message. Step 225 includes associating the third computing device 135 of the TGM 125 with the second computing device 140 of the PNA 120 and storing, in the connected database 130, a unique identifier indicating the association between the second computing device and the third computing device. The unique identifier may be generally defined as a specific attribute or value assigned to a piece of data or data record serving as a unique and unambiguous identifier for a particular piece of data. The unique identifier may be generated using random number generation algorithms, hash functions, concatenating, encrypting, salting, a combination of factors, etc. After the first computing device has associated the third computing device with the second computing device and stored the unique identifier in the connected database, the method will continue to step 230.

Step 230 includes receiving from the third computing device 135 of the TGM 125, a fourth message 340 containing PPA data 345. The data flow during this step is illustrated in FIG. 4 for reference. The PPA data may include contact information, personal information, relationship to the PNA, etc. In some embodiments, the fourth message may include one PPA's data, however in other embodiments the fourth message may include several PPAs' data. In other embodiments, the fourth message may be sent in the third message. In other embodiments, the third message may be sent before or after the fourth message. After the first computing device receives the fourth message containing PPA contact data, the method will continue to step 235.

Step 235 involves the first computing device sending a fifth message 350 which contains an invite 355 to join a trust group, to the fourth computing device 150 associated with the PPA 145. The data flow during this step is illustrated in FIG. 4 for reference. In some embodiments, the system may send a fifth message to a plurality of potential PPAs, whereas in other embodiments, the system may send a fifth message to one PPA at a time. The invite may be displayed across the user interface of the fourth computing device in the form of a text message, push notification, email, survey, phone call, app-based request, etc. In some embodiments, the invite may include PNA data. In other embodiments, the invite may only consist of a simple question such as “You're invited to join [insert PPA name/photo] trust group”; “Do you accept?”. Dependent on the response to the fifth message, the method will either stop or continue. If the PPA declines joining the trust group, the system may send the third computing device 135 a message notifying the TGM of the PPA refusal and return to step 230 awaiting a new potential PPA. If the fourth computing device accepts the invitation, the method will continue to step 240.

Step 240 includes receiving a sixth message 360 from the fourth computing device of the PPA containing an acceptance or denial 365 of the invite to join the trust group and at least one PPA availability profile 370. In some embodiments, if the PPA does not accept the invite, the system may send another message after a predetermined amount of time or may record the denial and move onto the next potential PPA. In other embodiments, the system may notify the TGM or the PNA of the denial. The data flow during this step is illustrated in FIG. 4 for reference. It is understood that the system includes at least one PPA allowing for the possibility of multiple PPAs. The PPA availability profile may be generally defined as a set of constraints that directly affect the PPA's ability to provide assistance to the PNA. This profile may include times or days the PPA is unavailable. For example, work hours or holidays may affect the PPA's ability to promptly provide assistance. In other embodiments, the PPA availability profile may include the PPA's physical location. Dependent upon what type of emergency the PNA is experiencing, the PPA may need to be close in distance to assist. After the sixth message is received, the method will continue to step 245.

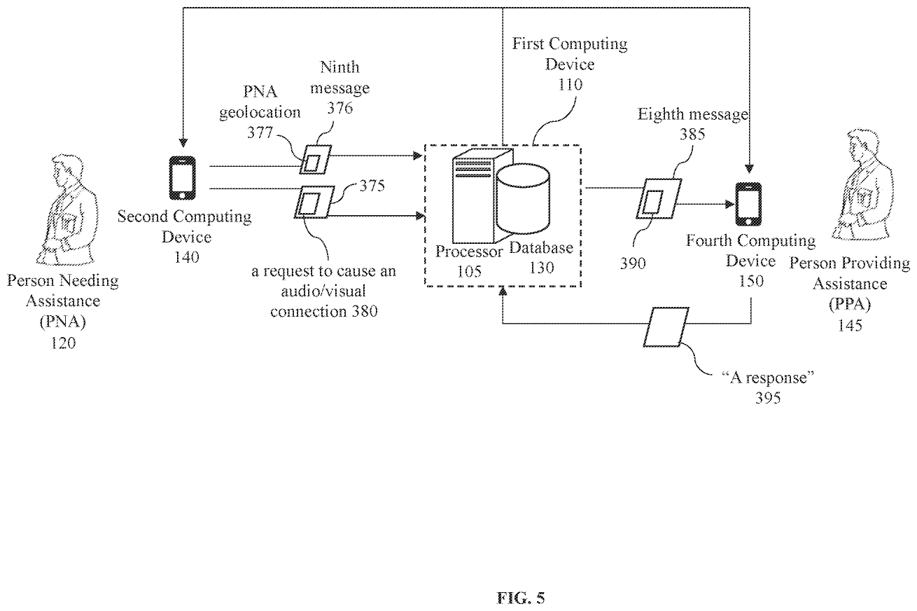

Step 245 includes storing the PPA availability profile in the PNA user record for each PPA user that accepts the invite to join the PPA group. In other embodiments, the PPA and TGM may each have their own user record in the connected database. After the PPAs availability profiles are stored in the PNA user record, the method will continue to step 250. Step 250 includes receiving, with the transceiver and from the second computing device 140, a seventh message 375 containing a request 380 to cause an audio-visual connection between the second computing device and the fourth computing device 150. The data flow during this step is illustrated in FIG. 5 for reference. An audio-visual connection may generally be described as phone calls, video calls, or photos and videos being sent from one device to another. This connection refers to the transmission of audio and video signals via network-based communication and software protocols. This type of connection is commonly referred to as a network-based or streaming connection. For reference, FIGS. 10B and 10C illustrate a computing device that has established a connection with another computing device and is visually communicating with one another. The type of connection may depend on the PNA and the PNAs abilities/disabilities. For example, a PNA having the sensory disability of blindness may prefer or require the use of an audio connection to communicate while also requiring a visual connection to allow the PPA to see for the PNA when in need of assistance. In another example embodiment, a PNA having the sensory disability of deafness may prefer or require a visual connection when in need of assistance. In other example embodiments, the PNA may be deaf and blind. In such an example, the PPA will likely utilize an audio and visual connection to observe the PNAs surroundings, however in order to communicate to the deafblind individual the computing device may need to transform the sound waves of the PPA to vibrations in such a manner that the PNA may understand. Other types of communication and connections may also be used and are within the spirit and the scope of the present invention.

In some embodiments, this request is the PNA's call for help. FIG. 10A shows an example embodiment of the second computing device's user interface displaying a button 141 that when pressed, will send a seventh message 375 containing a request 380 to cause an audio-visual connection between the second computing device and the fourth computing device 150. In other embodiments, the PNA may press a physical button on the phone to send the seventh message. Furthermore, in other example embodiments, the PNA's computing device may contain a virtual assistant such as Siri designed to interact with the user through voice recognition and natural language processing. In such example embodiments, the PNA may speak a certain predetermined phrase, such as “Siri call for assistance”, into the phone to initiate the seventh message and request for audio-visual connection between the second computing device and fourth computing device. After receiving the seventh message from the second computing device, the system will transmit a second audio-visual message on the second computing device comprising of a message of affirmation that the request has been transmitted. In some embodiments, the message of affirmation may be in the form of a voice recording of the TGM user or a selection of an audio-visual document. The message of affirmation may vary across other example embodiments. After the system, or first computing device 110, receives the request from the second computing device of the PNA, the method will continue to step 255.

Step 255 involves the first computing device creating a predetermined order in which to attempt to cause the audio-visual connection between the PPA and PNA based on the PPA's availability profile. However, prior to determining the predetermined order, the system will receive a ninth message 376 containing PNA geolocation data 377 from the second computing device 140, as shown in FIG. 5. In one embodiment, the PNA computing device may continually monitor the PNA geo-location and then send a message to the processor or the central or first computing device when the PNA computing device is outside a predetermined geographical constraint. The geographical constraints may be a function of any of the following: geographic location of the PNA user; geographic restrictions: date: geographic restrictions of the PPA user: calendar restrictions of the PPA, PNA, or TGM. In other embodiments, the PNA computing device may continually monitor the PNA computing device geo-location and continually send to the first computing device. However other embodiments of monitoring the geolocation of the PNA computing device may also be used and are within the spirit and the scope of the present invention. The system will then determine whether the streaming PNA geolocation data satisfies the geographic availability of at least one PPA's availability profile. As mentioned before, in step 230 the TGM will send the first computing device PPA data for one or more PPAs, next in step 235 an invitation to join the trust group is sent, and lastly in step 240 with the sixth message, the PPA provides its availability profile to be stored in the connected database. Therefore, the system, or first computing device 110, contains a plurality of PPA data and availability profiles, including geographic availability, for at least one or more PPAs. The first computing device may determine the order of which PPAs to call by comparing the profiles of each PPA and determining who is most available at the time the seventh and ninth message is received. In other embodiments, the system may send an eighth message to all available PPAs at the same time. For example, if the seventh message, or call for help, is received on a Tuesday at 3:00 pm, the system will compare all profiles to determine who is available at that time, for how long after that time, who/what device is in close proximity to the PNA device, and who is preferred by the PNA. For PNAs more likely to require physical assistance, the geolocation data may have a higher contribution when determining the order of PPAs called. Other factors may also contribute to the order in which the system will attempt to cause the audio-visual connection. After the order is determined, the method will continue to step 260.

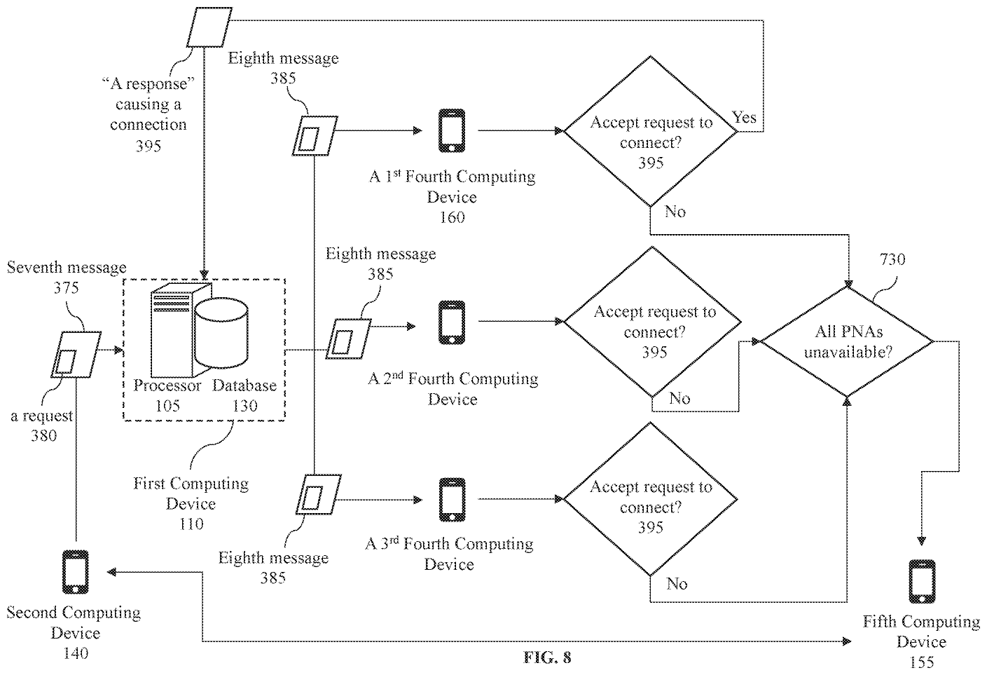

Step 260 includes sending, in the predetermined order, an eighth message comprising a request 390 to connect at least one fourth computing device 150 with the second computing device 140. The data flow during this step is illustrated in FIG. 5 for reference. Similar to the second message 320 referred to above, the request 390 may be sent via text message, SMS message, email, audio call, etc. The request may be displayed across the user interface in the form of a text message, push notification, email, survey, phone call, app-based request, etc. The fourth computing device may either accept or decline the request. This response 395 will determine if the connection is made or if another fourth computing device is contacted. If the response 395 contains a request approval, the method will continue to step 265. Step 265 is the audio-visual connection between the second and fourth computing devices. If response 395 contains a request denial, the method will repeat step 260 until all PPAs have been requested. If the system does not receive an approval from any PPA, the system will cause an audio-visual connection between the second computing device and a fifth computing device 155 of an emergency backup public crisis responder 146. If multiple people approve of the request, the system may connect the first person to accept. In other embodiments, the PPA's available may be displayed on the PNA's device allowing the PNA to choose which PPA to connect to. The data flow during this process is shown in FIG. 8 for reference.

FIG. 6 is a diagram showing a first subset group 175 of persons providing assistance and a second subset group 180 of persons providing assistance, according to an example embodiment. According to the example embodiment shown in FIG. 6, two subset groups each contain three PPAs, however in other embodiments, the number of subset groups and PPAs within the subsets may vary. These subsets may also be defined as the predetermined order mentioned above during step 255. These subsets may be organized in such a way that the PPAs are sorted by the likelihood of responding, for example, most likely to respond and least likely to respond. In other embodiments, the subsets may be randomized having likely and unlikely in the same subset. In other embodiments, the subsets may be based on availability profile parameters. For example, subset group 175 may include PPAs that are available after 5 pm, while subset group 180 may include PPAs that are available before 5 pm. Furthermore, each subset may be different as the PNA moves from one location to another.

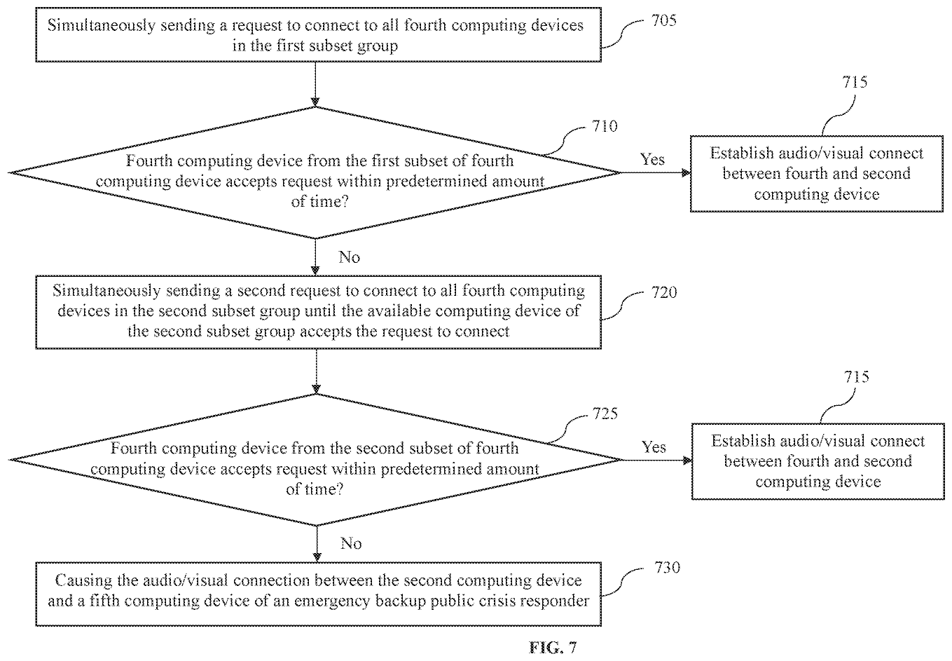

FIG. 7 is a flow chart showing the steps taken when attempting to establish an audio-visual connection between a person needing assistance and a person providing assistance, and FIG. 8 is a diagram showing the data flow when attempting to establish an audio-visual connection between the person needing assistance and a person providing assistance. As shown in FIG. 8, the PNA may first send a seventh message 375 or call, to the system or first computing device, requesting a connection. Once the request is made, the system will begin attempting to establish the audio-visual connection with at least one fourth computing device in the first subset and the second computing device of the PNA. As shown in FIG. 7, the method begins with step 705. Step 705 includes simultaneously sending an eighth message containing a request to connect to all fourth computing devices in the first subset group. After the eighth message 385 is sent to all fourth computing devices in the first subset group 175, the method continues to step 710.

Step 710 the processer of the first computing device determines if a fourth computing device from the first subset of fourth computing devices accepts the request within a predetermined amount of time. The predetermined amount of time may be generally defined as a specific duration that has been set, decided, or established in advance for a particular purpose or activity. More specifically, the predetermined amount of time may be determined by the TGM as a limit for how long the PNA should need to wait to receive assistance. If the PNA has a chronic health condition such as asthma, the predetermined amount of time may be fairly short (3-5 seconds) because of the likelihood of the PNA requiring emergency assistance or immediate help. In other example embodiments, the predetermined amount of time may be longer. For example, if the PNA has a mental disability such as anxiety or depression, where there is likely no immediate danger, the predetermined amount of time may provide the PPAs with a larger window to provide assistance. In other embodiments, the predetermined amount of time may vary and is within the spirit and the scope of the present invention.

If a fourth computing device accepts the request within the predetermined amount of time, then the response 395 will be sent back to the system, or first computing device 110, as shown in FIG. 8. The connection will then be made between the fourth computing device and the second computing device, as shown in step 715. If the first subset group fails to accept the request to connect, the method will continue to step 720.

In step 720 the system will attempt to establish a connection with the second subset group 180 of PPAs by simultaneously sending an eighth message containing a second request to connect to all fourth computing devices in the second subset group. After the eighth message is sent to all fourth computing devices in the second subset group, the method continues to step 725. Step 725 determines if a fourth computing device from the second subset of fourth computing devices accepts the request within a predetermined amount of time. As mentioned above the predetermined amount of time may vary dependent on the PNA or TGM. If a fourth computing device accepts the request within the predetermined amount of time, then the acceptance response will be sent back to the system and the connection will then be made between the fourth computing device and the second computing device, as shown in step 715. If the second subset group fails to accept the request to connect, then the method will continue to step 730.

In step 730, the system will cause an audio-visual connection between the second computing device of the PNA and a fifth computing device 155 of an emergency backup public crisis responder 146. The emergency backup public crisis responder may generally be defined as an individual or team serving as a secondary support resource to the PNA and trust group. Examples of emergency backup public crisis responders may include police officers, emergency medical technicians (EMTs), psychologists, counselors, crisis hotline operators, etc.

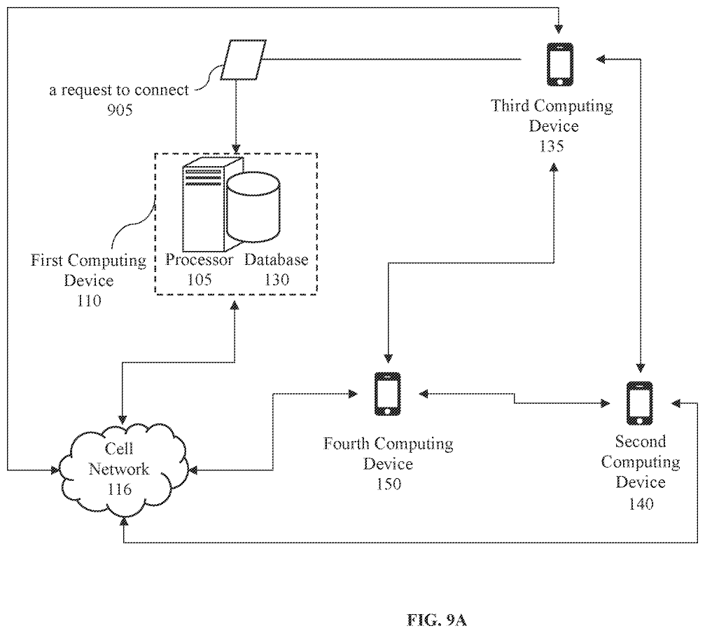

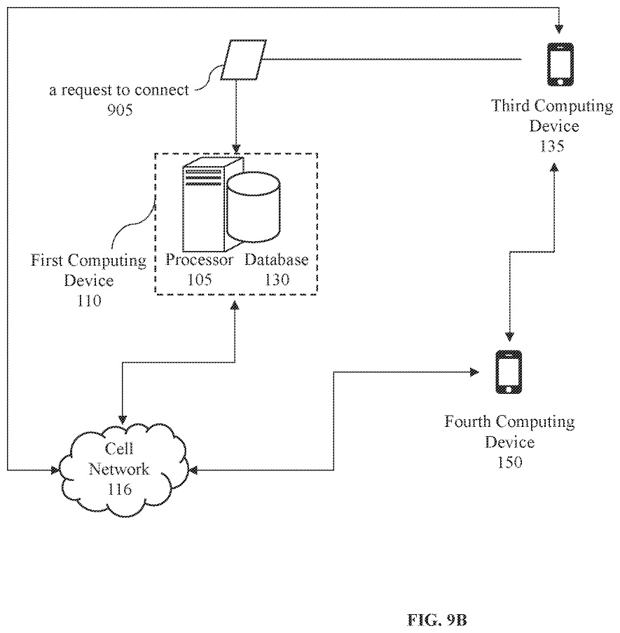

FIG. 9A is a diagram showing the data flow as the system receives a request to connect 905 the third computing device 135 associated with the TGM 125, the second computing device 140 associated with the PNA 120, and the fourth computing device 150 associated with the PPA 145 to each other. In the present example embodiment, the request is made by the third computing device, however, in other embodiments, the request to connect may be made by the TGM, the PNA, the PPA, third-party management, etc. In an example embodiment, the request may be made for purposes such as group meetings in preparation for days or times when the PNA is most likely to need assistance. For example, if a visually impaired person is visiting a new restaurant or city, the support team may want to form an audio-visual connection to discuss who is available or who is better skilled to handle a situation where the PNA requires assistance. In other example embodiments, the request to connect may be made to introduce a new PPA to the trust group.

FIG. 9B is a diagram showing the data flow as the system receives a request to connect 905 the third computing device 135 associated with the TGM 125 and the fourth computing device 150 associated with the PPA 145 to each other. In the present example embodiment, the request is made by the third computing device, however, in other embodiments, the request to connect may be made by the TGM, a PPA, third-party management, etc. In an example embodiment, the request may be made to discuss matters that do not need the attention of the PNA. For example, gift-giving or surprise gatherings may be planned by the TGM and PPAs for the PNA. In such an example, the TGM and the PPAs would form an audio-visual connection to discuss details together. In other embodiments, the TGM and PPAs may want to discuss possible situations that may cause the PNA to require assistance. For example, if the PNA is diagnosed with a mental disability such as panic attacks and is going on a date, the TGM and PPAs may want to discuss the details or likelihood of needing to assist without the PNA getting more anxious or scared leading up to the date. In other example embodiments, the request may be made for other purposes and is within the spirit and the scope of the present invention.

FIG. 11 is a block diagram of a system including an example computing device 1100 and other computing devices. Consistent with the embodiments described herein, the aforementioned actions performed by processor 105 or computing devices 110, 140, 135, 150, 155, and 160 may be implemented in a computing device, such as the computing device 1100 of FIG. 11. Any suitable combination of hardware, software, or firmware may be used to implement the computing device 1100. The aforementioned system, device, and processors are examples and other systems, devices, and processors may comprise the aforementioned computing device. Further-more, computing device 1100 may comprise an operating environment for the methods shown in FIGS. 1 through 10 above.

With reference to FIG. 11, a system consistent with an embodiment of the invention may include a plurality of computing devices, such as computing device 1100. In a basic configuration, computing device 1100 may include at least one processing unit 1102 and a system memory 1104. Depending on the configuration and type of computing device, system memory 1104 may comprise, but is not limited to, volatile (e.g., random access memory (RAM)), nonvolatile (e.g., read-only memory (ROM)), flash memory, or any combination or memory. System memory 1104 may include operating system 1105, one or more programming modules 1106 (such as program module 1107). Operating system 1105, for example, may be suitable for controlling computing device 1100's operation. In one embodiment, programming modules 1106 may include, for example, a program module 1107. Furthermore, embodiments of the invention may be practiced in conjunction with a graphics library, other operating systems, or any other application program and is not limited to any particular application or system. This basic configuration is illustrated in FIG. 11 by those components within a dashed line 1120.

Computing device 1100 may have additional features or functionality. For example, computing device 1100 may also include additional data storage devices (removable and/or non-removable) such as, for example, magnetic disks, optical disks, or tape. Such additional storage is illustrated in FIG. 11 by a removable storage 1109 and a non-removable storage 1110. Computer storage media may include volatile and nonvolatile, removable, and non-removable media implemented in any method or technology for storage of information, such as computer readable instructions, data structures, program modules, or other data. System memory 1104, removable storage 1109, and non-removable storage 1110 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information, and which can be accessed by computing device 1100. Any such computer storage media may be part of device 1100. Computing device 1100 may also have input device(s) 1112 such as a keyboard, a mouse, a pen, a sound input device, a camera, a touch input device, etc. Output device(s) 1114 such as a display, speakers, a printer, etc. may also be included. The aforementioned devices are only examples, and other devices may be added or substituted.

Computing device 1100 may also contain a communication connection 1116 that may allow device 1100 to communicate with other computing devices 1118, such as over a network in a distributed computing environment, for example, an intranet or the Internet. Communication connection 1116 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acous-tic, radio frequency (RF), infrared, and other wireless media. The term computer readable media as used herein may include both computer storage media and communication media.

As stated above, a number of program modules and data files may be stored in system memory 1104, including operating system 1105. While executing on processing unit 1102, programming modules 1106 may perform processes including, for example, one or more of the methods shown in FIGS. 3 through 7 above. Computing device 1100 may also include a graphics processing unit, which supplements the processing capabilities of processor 1102 and which may execute programming modules 1106, including all or a portion of those processes and methods shown in FIGS. 1 through 10 above. The aforementioned processes are examples, and processing units 1102 may perform other processes. Other program-ming modules that may be used in accordance with embodi-ments of the present invention may include electronic mail and contacts applications, word processing applications, spreadsheet applications, database applications, slide presentation applications, drawing or computer aided application programs, etc.

Generally, consistent with embodiments of the invention, program modules may include routines, programs, components, data structures, and other types of structures that may perform particular tasks or that may implement particular abstract data types. Moreover, embodiments of the invention may be practiced with other computer system configura-tions, including handheld devices, multiprocessor systems, microprocessor based or programmable consumer electronics, minicomputers, mainframe computers, and the like. Embodiments of the invention may also be practiced in distributed computing environments where tasks are performed by remote processing devices that are linked through a communications network. In a distributed computing environment, program modules may be located in both local and remote memory storage devices.

Furthermore, embodiments of the invention may be prac-ticed in an electrical circuit comprising discrete electronic elements, packaged or integrated electronic chips containing logic gates, a circuit utilizing a microprocessor, or on a single chip (such as a System on Chip) containing electronic elements or microprocessors. Embodiments of the invention may also be practiced using other technologies capable of performing logical operations such as, for example, AND, OR, and NOT, including but not limited to mechanical, optical, fluidic, and quantum technologies. In addition, embodiments of the invention may be practiced within a general-purpose computer or in any other circuits or systems.

Embodiments of the present invention, for example, are described above with reference to block diagrams and/or operational illustrations of methods, systems, and computer program products according to embodiments of the inven-tion. The functions/acts noted in the blocks may occur out of the order as shown in any flowchart. For example, two blocks shown in succession may in fact be executed substantially concurrently or the blocks may sometimes be executed in the reverse order, depending upon the function-ality/acts involved.

While certain embodiments of the invention have been described, other embodiments may exist. Furthermore, although embodiments of the present invention have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the invention.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A computer implemented method, executable on at least one processor of a first computing device, for generating personalized responsive assistance for a person needing assistance (PNA) by a set of persons providing assistance (PPA) who are personally known by the person needing assistance, wherein the computer implemented method comprises:
a. receiving, with a transceiver of the first computing device, over a communications network, a first message comprising PNA contact data for a PNA user and a trust group manager (TGM) contact data of a TGM user;
b. generating a PNA user record for the PNA user and storing, in a connected database, the PNA contact data;
c. sending, over the communications network, to a third computing device associated with the TGM user, a second message comprising a first request for an approval to associate the third computing device of the TGM user with a second computing device of the PNA user;
d. receiving, over the communications network, from the third computing device, a third message comprising the approval of the first request to associate the third computing device of the TGM user with the second computing device of the PNA user;
e. after receiving to the third message, associating the third computing device of the TGM user with the second computing device of the PNA user and storing, in the connected database, a unique identifier indicating an association between the second computing device and the third computing device;
f. receiving, from the third computing device of the TGM user, a fourth message comprising PPA data for at least one PPA user, wherein the PPA data comprises contact information for the at least one PPA user;
g. sending, over the communications network to at least one fourth computing device of the at least one PPA user, a fifth message comprising an invite to join a trust group associated with the PNA user record;
h. receiving a sixth message from the at least one fourth computing device in response to the invite to join the trust group, wherein the sixth message comprises an acceptance of the invite to join the trust group and at least one PPA availability profile;
i. storing the at least one PPA availability profile in the PNA user record for each PPA user that accepted the invite to join the trust group;
j. receiving, with the transceiver, from the second computing device, a seventh message comprising a second request to cause an audio-visual connection between the second computing device with the at least one fourth computing device of the at least one PPA user;
k. determining, based on the at least one PPA availability profile, a predetermined order in which to attempt to cause the audio-visual connection between the second computing device and the at least one fourth computing device of the at least one PPA user;
l. sending in the predetermined order, over the communications network to the at least one fourth computing device, an eighth message, wherein the eighth message comprises a third request to connect the at least one fourth computing device with the second computing device; and
m. causing the audio-visual connection between the second computing device and an available computing device of the at least one fourth computing device.

2. The computer implemented method of claim 1 wherein the predetermined order for causing the audio-visual connection between the second computing device and the at least one fourth computing device of the at least one PPA user comprises:
a. a first subset group;
b. at least one second subset group; and
c. wherein the computer implemented method further comprises,
i. attempting to establish the audio-visual connection with the at least one fourth computing device in the first subset group by simultaneously sending the third request to connect to a first subset of fourth computing devices of the at least one fourth computing device in the first subset group until the available computing device of the first subset group accepts the third request to connect; and
ii. if the audio-visual connection between the first subset of fourth computing devices fails to accept the third request to connect after a predetermined amount of time, then attempting to establish the audio-visual connection with a second subset of fourth computing devices of the at least one fourth computing device in the at least one second subset group by simultaneously sending the third request to connect to the second subset of fourth computing devices until the available computing device of the at least one second subset group accepts the third request to connect.

3. The computer implemented method of claim 2 further comprising, wherein if the audio-visual connection between the second computing device fails to connect to the at least one fourth computing device from the first subset group and the at least one second subset group, then causing the audio-visual connection between the second computing device and a fifth computing device of an emergency backup public crisis responder.

4. The computer implemented method of claim 1 further comprising:
a. receiving a fourth request to connect the third computing device to the at least one fourth computing device and the second computing device; and
b. causing a second audio-visual connection between the third computing device, and the at least one fourth computing device and the second computing device.

5. The computer implemented method of claim 1 further comprising:
a. receiving a fifth request to connect the third computing device the at least one fourth computing device and the third computing device; and
b. causing a third audio-visual connection between the third computing device, and the at least one fourth computing device.

6. The computer implemented method of claim 1 wherein the at least one PPA availability profile comprises at least one geographic availability.

7. The computer implemented method of claim 6 further comprising:
a. receiving a ninth message comprising a streaming PNA geolocation data from the second computing device; and
b. prior to determining, based on the at least one PPA availability profile, the predetermined order in which to attempt to cause the audio-visual connection, determining, with the at least one processor, whether the streaming PNA geolocation data satisfies the at least one geographic availability of the at least one PPA availability profile.

8. The computer implemented method of claim 1 further comprising:
a. receiving a plurality of ad requests from a sixth computing device, based on the PNA user record, wherein the PNA user record comprises at least one TGM indicated advertising preference, and
b. displaying a plurality of advertisements, based on the plurality of ad requests, on the at least one fourth computing device associated with the at least one PPA user.

9. The computer implemented method of claim 1 further comprising:
a. after receiving the seventh message from the second computing device, transmitting a second audio-visual message on the second computing device, wherein the second audio-visual message comprises an affirmation message that the third request to connect the at least one fourth computing device with the second computing device has been transmitted.

10. The computer implemented method of claim 9 further comprising:
a. receiving the affirmation message from the third computing device associated with the TGM user, wherein the affirmation message is at least one of (i) a voice recording of the TGM user, and (ii) a selection of an audio-visual document.

11. A computer implemented method, executable on at least one processor of a first computing device, for generating personalized responsive assistance for a person needing assistance (PNA) by a set of persons providing assistance (PPA) who are personally known by the person needing assistance, wherein the computer implemented method comprises:
a. receiving, with a transceiver of the first computing device, over a communications network, a first message from a second computing device, the first message comprising PNA contact data for a PNA user;
b. receiving, with the transceiver, at least one second message from at least one third computing device, the at least one second message comprising PPA data for at least one PPA user, wherein the PPA data comprises contact information for the at least one PPA user and an availability profile;
c. generating a PNA user record for the PNA user and at least one PPA user record for the at least one PPA user and storing the PNA user record and the at least one PPA user record in a connected database;
d. associating the at least one PPA user record with the PNA user record;
e. receiving, with the transceiver, from the second computing device, a third message comprising a first request to cause an audio-visual connection between the second computing device with the at least one third computing device;
f. determining, based on the availability profile of each PPA user record of the at least one PPA user record, a predetermined order in which to attempt to cause the audio-visual connection between the second computing device and the at least one third computing device of the at least one PPA user;
g. sending in the predetermined order, over the communications network to the at least one third computing device, a fourth message comprising a second request to connect the at least one third computing device with the second computing device; and
h. causing the audio-visual connection between the second computing device and an available computing device of one of the at least one third computing device based on a response from the available computing device of the at least one third computing device.

12. The computer implemented method of claim 11 wherein the predetermined order for causing the audio-visual connection between the second computing device and the at least one third computing device of the at least one PPA user comprises:
a. a first subset group;
b. at least one second subset group; and
c. wherein the computer implemented method further comprises,
i. attempting to establish the audio-visual connection with the at least one third computing device in the first subset group by simultaneously sending the second request to connect to a first subset of third computing devices of the at least one third computing device in the first subset group until the available computing device of the first subset group accepts the second request to connect; and
ii. if the audio-visual connection between the first subset of third computing devices fails to accept the second request to connect after a predetermined amount of time, then attempting to establish the audio-visual connection with a second subset of third computing devices of the at least one third computing device in the at least one second subset group by simultaneously sending the second request to connect to the second subset of third computing devices until the available computing device of the at least one second subset group accepts the second request to connect.

13. The computer implemented method of claim 12 wherein the availability profile comprises at least one geographic availability.

14. The computer implemented method of claim 13 further comprising:
a. receiving a fifth message comprising a streaming PNA geolocation data from the second computing device; and
b. prior to determining, based on the availability profile of the at least one PPA user record, the predetermined order in which to attempt to cause the audio-visual connection, determining, with the at least one processor, whether the streaming PNA geolocation data satisfies the at least one geographic availability of the availability profile of the at least one PPA user record.

15. The computer implemented method of claim 14 further comprising:
a. after receiving the third message from the second computing device, transmitting a second audio-visual message on the second computing device, wherein the second audio-visual message comprises a message of affirmation that the second request to connect the at least one third computing device with the second computing device has been transmitted.

16. The computer implemented method of claim 15 further comprising wherein at least one of (i) the PNA user record and (ii) a PPA user record of the at least one PPA user record defines a trust group manager (TGM) user record.

17. A computer implemented method, executable on at least one processor of a first computing device, for generating personalized responsive assistance for a person needing assistance (PNA) by a set of persons providing assistance (PPA) who are personally known by the person needing assistance, wherein the computer implemented method comprises:
a. receiving, with a transceiver of the first computing device, over a communications network, a first message comprising PNA contact data for a PNA user and a trust group manager (TGM) contact data of a TGM user;
b. generating a PNA user record for the PNA user and storing, in a connected database, the PNA contact data;
c. sending, over the communications network, to a third computing device associated with the TGM user, a second message comprising a first request for an approval to associate the third computing device of the TGM user with a second computing device of the PNA user;
d. receiving, over the communications network, from the third computing device, a third message comprising the approval of the first request to associate the third computing device of the TGM user with the second computing device of the PNA user;
e. in response to the third message, associating the third computing device of the TGM user with the second computing device of the PNA user and storing, in the connected database, a unique identifier indicating an association between the second computing device and the third computing device;
f. receiving, from the third computing device of the TGM user, a fourth message comprising PPA data for at least one PPA user, wherein the PPA data comprises contact information for the at least one PPA user;
g. sending, over the communications network to at least one fourth computing device of the at least one PPA user, a fifth message comprising an invite to join a trust group associated with the PNA user record;
h. receiving a sixth message from the at least one fourth computing device in response to the invite to join the trust group, wherein the sixth message comprises an acceptance of the invite to join the trust group and at least one PPA availability profile;
i. wherein the at least one PPA availability profile comprises at least one geographic availability;
i. storing the at least one PPA availability profile in the PNA user record for each PPA user that accepted the invite to join the trust group;
j. receiving, with the transceiver, from the second computing device, a seventh message comprising a second request to cause an audio-visual connection between the second computing device with the at least one fourth computing device of the at least one PPA user;
k. receiving streaming PNA geolocation data from the second computing device;
l. determining, with the at least one processor, whether the streaming PNA geolocation data satisfies the at least one geographic availability of the at least one PPA availability profile;
m. determining, based on the at least one PPA availability profile, a predetermined order in which to attempt to cause the audio-visual connection between the second computing device and the at least one fourth computing device of the at least one PPA user;
i. wherein the predetermined order for causing the audio-visual connection between the second computing device and the at least one fourth computing device of the at least one PPA user comprises:
a. a first subset group; and
b. at least one second subset group;
n. sending in the predetermined order, over the communications network to the at least one fourth computing device, an eighth message, wherein the eighth message comprises a third request to connect the at least one fourth computing device with the second computing device;
o. attempting to establish the audio-visual connection with the at least one fourth computing device in the first subset group by simultaneously sending the third request to connect to a first subset of fourth computing devices of the at least one fourth computing device in the first subset group until an available computing device of the first subset group accepts the third request to connect;
p. if the audio-visual connection between the first subset of fourth computing devices fails to accept the third request to connect after a predetermined amount of time, then attempting to establish the audio-visual connection with a second subset of fourth computing devices of the at least one fourth computing device in the at least one second subset group by simultaneously sending the third request to connect to the second subset of fourth computing devices until the available computing device of the at least one second subset group accepts the third request to connect; and
q. causing the audio-visual connection between the second computing device and the available computing device of one of the at least one fourth computing device based on a response from the available computing device of the at least one fourth computing device in the first subset group and the at least one second subset group.

18. The computer implemented method of claim 17 further comprising, wherein if the audio-visual connection between the second computing device fails to connect to the at least one fourth computing device from the first subset group and the at least one second subset group, then causing the audio-visual connection between the second computing device and a fifth computing device of an emergency backup public crisis responder.

19. The computer implemented method of claim 18 further comprising:
a. receiving a fourth request to connect the third computing device to the at least one fourth computing device and the second computing device; and
b. causing a second audio-visual connection between the third computing device, and the at least one fourth computing device and the second computing device.

20. The computer implemented method of claim 19 further comprising:
a. receiving a fifth request to connect the third computing device the at least one fourth computing device and the third computing device; and
b. causing a third audio-visual connection between the third computing device, and the at least one fourth computing device.
